(19) United States
(12) Patent Application Publication
(10) Pub. No.: US 2012/0296364 A1
(43) Pub. Date: Nov. 22, 2012

(54) BALLOON CATHETER

(75) Inventors: BENJAMIN GUNDALE, PLYMOUTH, MN (US); JEFFRY JOHNSON, PLYMOUTH, MN (US); CHUANJING XU, CHASKA, MN (US)

(73) Assignee: BOSTON SCIENTIFIC SCIMED, INC., MAPLE GROVE, MN (US)

(21) Appl. No.: 13/472,224

(22) Filed: May 15, 2012

Related U.S. Application Data
(60) Provisional application No. 61/488,482, filed on May 20, 2011.

(51) Int. Cl. A61M 29/02; B32B 37/12; B32B 37/06; B23P 11/00

(52) U.S. Cl. 606/192; 29/428; 156/60; 156/84

(57) ABSTRACT

Balloon catheter and methods for making and using balloon catheters are disclosed. An example balloon catheter may include a proximal shaft. A midshaft may be attached to the proximal shaft. The midshaft may have an outer wall. A distal shaft may be attached to the midshaft. A balloon may be coupled to the distal shaft. An inflation lumen may be defined that extends from the proximal shaft, through the midshaft, and into the distal shaft. The inflation lumen may be in fluid communication with the balloon. A support member may be attached to the outer wall of the midshaft.

### CROSS-REFERENCE TO RELATED APPLICATION

This application claims priority under 35 U.S.C. §119 to U.S. Provisional Application Ser. No. 61/488,482, filed May 20, 2011, the entirety of which is incorporated herein by reference.

### TECHNICAL FIELD

The present invention relates generally to catheters for performing medical procedures. More particularly, the present invention relates to balloon catheters.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device may include a balloon catheter. An example balloon catheter may include a proximal shaft. A midshaft may be attached to the proximal shaft. The midshaft may have an outer wall. A distal shaft may be attached to the midshaft. A balloon may be coupled to the distal shaft. An inflation lumen may be defined that extends from the proximal shaft, through the midshaft, and into the distal shaft. The inflation lumen may be in fluid communication with the balloon. A support member may be attached to the outer wall of the midshaft.

Another example balloon catheter may include a proximal shaft. A midshaft may be attached to the proximal shaft. The midshaft may have an outer wall. A distal shaft may be attached to the midshaft. A balloon may be coupled to the distal shaft. A guidewire port may be defined in the midshaft that provides access to a guidewire lumen. A support member may be attached to the outer wall of the midshaft. The support member may include a longitudinally extending rod with a proximal end disposed proximally of the guidewire port and a distal end disposed distally of the guidewire port.

An example method for manufacturing a balloon catheter may include providing a catheter shaft. The catheter shaft may include a proximal shaft, a midshaft attached to the proximal shaft, and a distal shaft attached to the midshaft. A guidewire port may be defined in the midshaft. The guidewire port may provide access to a guidewire lumen formed in the catheter shaft. The method may also include providing a support member and attaching the support member to the midshaft. The support member may extend both proximally and distally of the guidewire port.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example balloon catheter;

FIG. 2 is a cross-sectional view of a portion of the example balloon catheter shown in FIG. 1;

FIG. 3 is a cross-sectional view taken through line 3-3 in FIG. 2;

FIG. 4 is a side view of a portion of the example balloon catheter shown in FIGS. 1-4;

FIG. 5 is a cross-sectional view taken through line 5-5 in FIG. 4; and

FIGS. 6-11 illustrate some of the example method steps for manufacturing the balloon catheter shown in FIG. 1-5.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

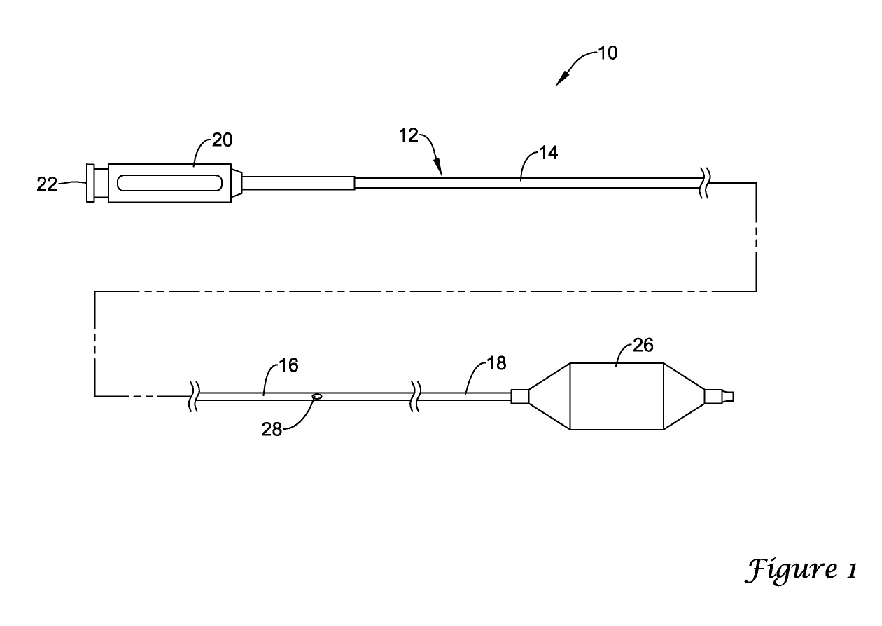

FIG. 1 is a plan view of an example catheter 10, for example a balloon catheter. Catheter 10 may include a catheter shaft 12 having a proximal shaft portion 14, a midshaft portion 16 and a distal shaft portion 18. In some embodiments, proximal shaft portion 14 may be a metallic hypotube. Midshaft portion 16 may be fitted over, fitted within, or abut proximal shaft portion 14, as appropriate. Likewise, distal shaft portion 18 may be fitted over, fitted within, or abut midshaft portion 16. These are just examples as any suitable arrangement may be utilized. A hub 20 may be attached to proximal shaft portion 14. Hub 20 may include one or more ports such as, for example, a port 22.

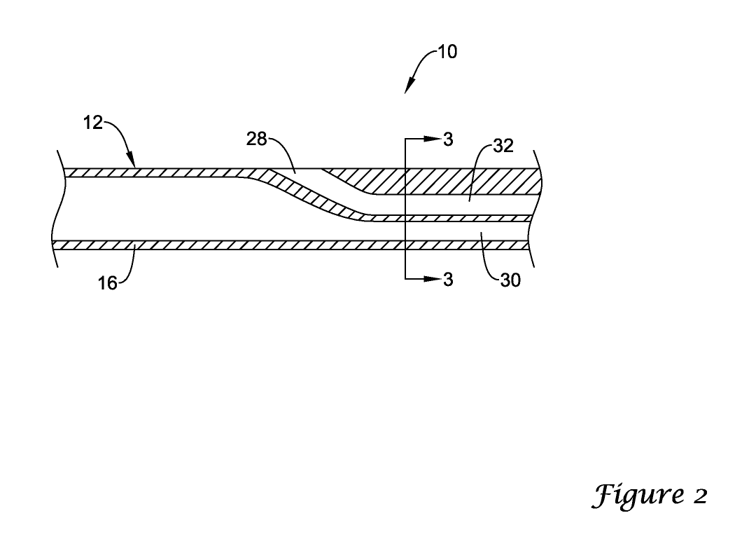

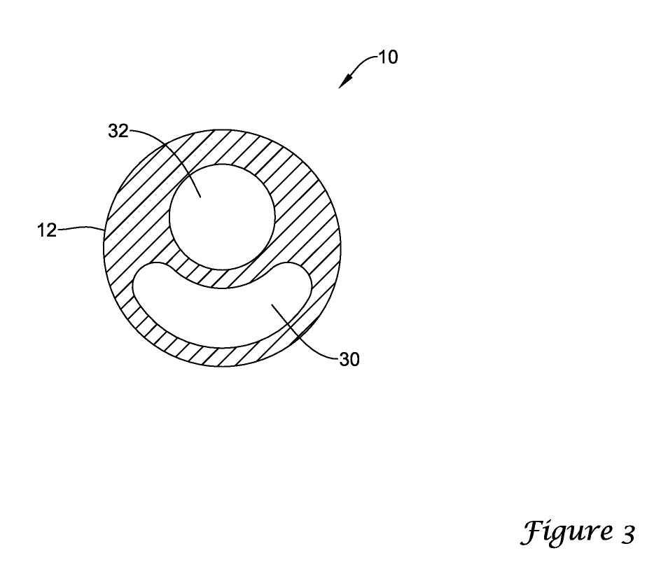

An expandable balloon 26 may be attached to distal shaft portion 18. Balloon 26 may be expanded by infusing inflation media through an inflation lumen 30, which is shown in FIG. 2. In at least some embodiments, port 22 may provide access to inflation lumen 30. Accordingly, a suitable inflation device may be attached to port 22 and inflation media may be passed through inflation lumen 30 to inflate balloon 26. Along a region of midshaft portion 16, inflation lumen 30 may have an annular shape as seen in FIG. 3. This may be due to the formation of a guidewire port 28 in midshaft portion 16. Some additional details regarding the formation of guidewire port 28 and/or inflation lumen 30 are provided herein.

As indicated above, guidewire port 28 may be formed in midshaft portion 16. For example, guidewire port 28 may be an opening extending through the wall of midshaft portion 16 that provides access to a guidewire lumen 32. In the embodiment depicted in FIG. 2, guidewire port 28 is positioned at a location that is distal to the proximal end of catheter shaft 12. When so arranged, catheter 10 may be a single-operator-exchange or rapid-exchange catheter, which allows catheter 10 to be used with a shorter guidewire. As such, guidewire lumen 32 may extend over only a portion of the length of catheter shaft 12. For example, guidewire lumen 32 may extend along distal shaft portion 18 and part of midshaft portion 16. Other embodiments, however, are contemplated where catheter 10 is an over-the-wire catheter or fixed wire catheter. In these embodiments, guidewire lumen 32 may extend along essentially the entire length of catheter shaft 12.

Catheters like catheter 10 may be designed to have increased or increasing distal flexibility. This may be desirable because portions of the catheter 10, particularly distal portions, may need to navigate sharp bends or turns within the vasculature. When the catheter shaft includes multiple sections or portions, however, the transition points between the sections may have a tendency to be more susceptible to kinking or buckling. For example, the transition point or points where the catheter shaft (e.g., catheter shaft 12) transitions from a relatively stiff proximal shaft portion (e.g., proximal shaft portion 14, which may take the form of a hypotube) to a more flexible midshaft and/or distal portion (e.g., midshaft portion 16 and/or distal shaft portion 18) may be susceptible to kinking and/or buckling.

In addition, because more distal portions of the catheter 10 may be designed to be highly flexible, it may be challenging to push the catheter through the vasculature in a reliable manner. In other words, increased distal flexibility, while being desirable for allowing the catheter to navigate the tortuous anatomy, may make it more difficult to “push” the catheter through the anatomy.

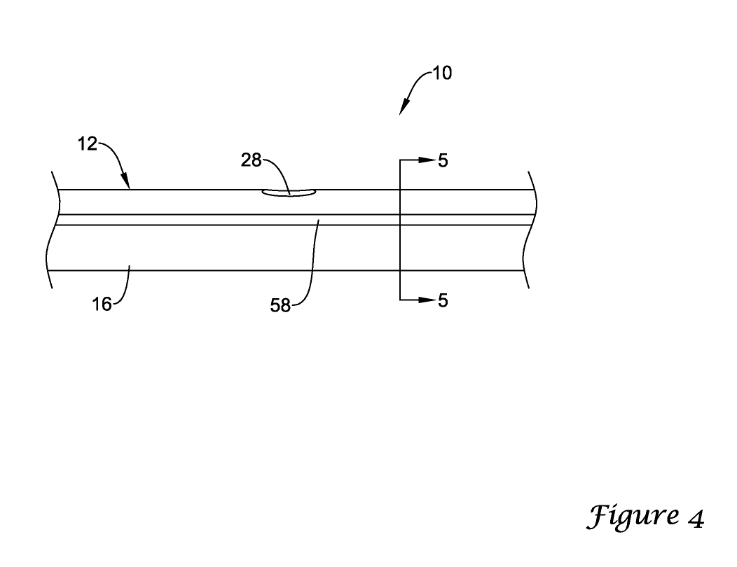

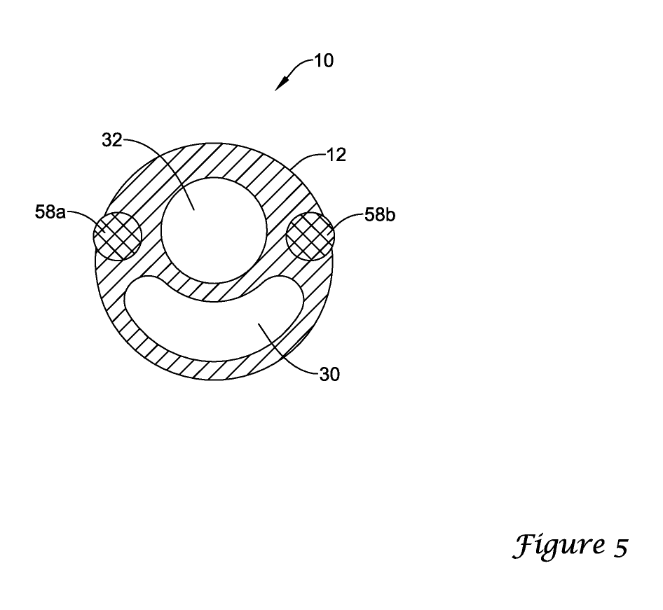

In order to improve the transition in flexibility from proximal shaft portion 14 to midshaft portion 16 and/or distal shaft portion 18 and in order to make catheter shaft 12 more “pushable” through the anatomy, catheter shaft 12 may include a support member 58 as shown in FIGS. 4-5. Support member 58 may take the form of a wire or rod that extends along a portion of catheter shaft 12. In at least some embodiments, support member 58 may extend along midshaft portion 16. This may include extending along a region of midshaft portion 16 (e.g., extending distally of guidewire port 28, proximally of guidewire port 28, or both) or along essentially the entire length of midshaft portion 16. Support member 58 may also extend along a region of proximal shaft portion 14 and/or along a region of distal shaft portion 18.

Support member 58 may desirably provide structural support that may ease or otherwise smooth the transition in flexibility from proximal shaft portion 14 to midshaft portion 16 and/or distal shaft portion 18. This may reduce the chances that catheter shaft 12 may kink or buckle, particularly at these locations. In addition, because midshaft portion 16 and/or distal shaft portion 18 are more flexible than proximal shaft portion 14, support member 58 may also provide additional structural support that improves the “pushability” of catheter shaft 12.

The form and configuration of support member 58 may vary considerably. For the purposes of this disclosure, support member 58 may be understood to be a structure or feature incorporated onto catheter shaft 12 that is different than other support structures that may be incorporated directly into the wall of catheter shaft 12 such as braids or coils. For example, support member 58 may be a generally linear or longitudinally extending shaft that is disposed along a discrete portion of the circumference of catheter shaft 12 (unlike braids or coils, which tend to extend about the complete circumference of a shaft). In other words, support member 58 may be a structure that is different from a supporting braid or coil that is directly incorporated into the wall of catheter shaft 12. Indeed, in at least some embodiments, catheter shaft 12 may include such supporting structure or structures directly within the wall of catheter shaft 12 (e.g., a coil, braid, etc.) in addition to support member 58.

Any suitable number of support members 58 may be utilized in order to provide the desired support to catheter shaft 12. For example, FIG. 5 illustrates that catheter shaft 12 may include a pair of support members 58a/58b disposed on opposite sides of catheter shaft 12. This, however, is not intended to be limiting as any suitable number (e.g., 1, 2, 3, 4, 5, 6, 7, 8, 9, 10, or more) of support members 58 may be utilized and these support members 58 may be arranged in any suitable manner (regularly around catheter shaft 12, evenly around catheter shaft 12, unevenly around catheter shaft 12, or in any suitable manner).

Support member 58 may be attached to catheter shaft 12 (e.g., midshaft portion 16) in any suitable manner. For example, support member 58 may be bonded to the outer surface of midshaft portion 16 with an adhesive. Alternatively, support member 58 may be thermally bonded to midshaft portion 16. This may include, for example, disposing support member 58 along midshaft portion 16, disposing a sleeve or jacket (e.g., a heat shrink tube) about midshaft portion 16 and support member 58, and heating the heat shrink tube to compress catheter shaft 12 and, as desired, embed or partially embed support member 58 in the wall of, for example, midshaft portion 16. Heating may include heating with a laser or any suitable heat source.

When attached to catheter shaft 12, support member 58 may be disposed along the exterior or outer wall of midshaft portion 16. In some of these and other embodiments, support member 58 may be partially embedded within the wall of midshaft portion 16. In still other embodiments, support member 58 may be completely embedded within the wall of midshaft portion 16 (e.g., the outer surface of support member 58 is located underneath the outer surface of midshaft portion 16). If catheter shaft 12 includes more than one support member 58, the support members 58 may all be attached in the same manner (e.g., along the exterior, partially embedded, completely embedded, etc.) or in different manners.

While support member 58 is described as being a rod or wire, this also is not intended to be limiting. In general, at least some embodiments of support member 58 may be elongate or substantially “straight” wires, rods, or tubes. The rods may include a single wire or filament or the rods may include a plurality of filaments. In embodiments that include more than one filament, the filaments may be longitudinally aligned, arranged in a helical manner, twisted, braided, or arranged in any suitable manner.

The materials used for support member 58 may include a metal. This may include any of the metals, to the extent appropriate, disclosed herein. For example, support member 58 may include stainless steel, nickel-titanium alloy, or the like. In other embodiments, support member 58 may include a relatively stiff polymer. This may include any of the polymers, to the extent appropriate, disclosed herein. For example, support member 58 may include polyimide, polyetheretherketone, or any other suitable material.

Catheter 10 may also include other structures that may be commonly associated with catheters. For example, a core wire (not shown) may be disposed within a portion of inflation lumen 30. The core wire may extend across midshaft portion 16 and may further improve the transition in flexibility along the length of catheter shaft 12 and/or improve catheter pushability. In addition, catheter 10 may include one or more radiopaque markers or bands, which may aid in fluoroscopically imaging catheter 10. These are just examples.

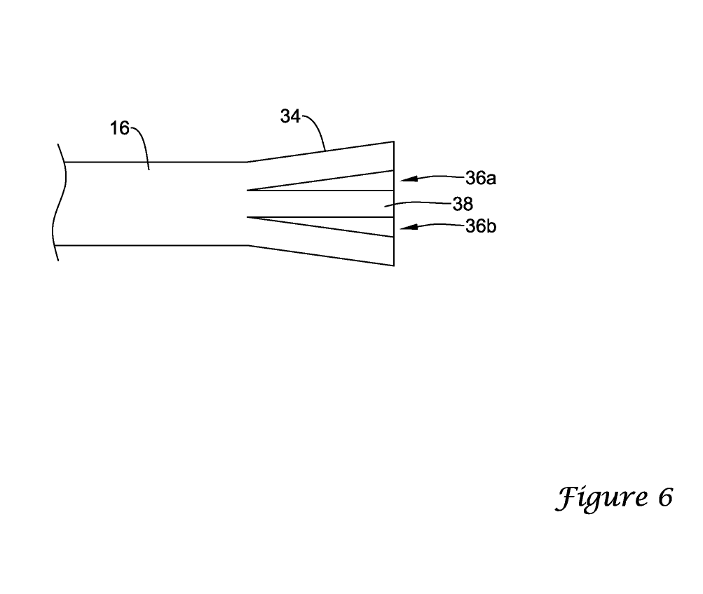

FIGS. 6-11 illustrate some of the processing steps that may be utilized to form catheter 10 and/or catheter shaft 12. For example, FIG. 6 shows part of midshaft portion 16. Here it can be seen that a distal end 34 of midshaft portion 16 may be flared or otherwise enlarged. In addition, one or more cuts or slots, for example cuts 36a/36b, may be formed in distal end 34 of midshaft portion 16. A tongue 38 may be defined between cuts 36a/36b.

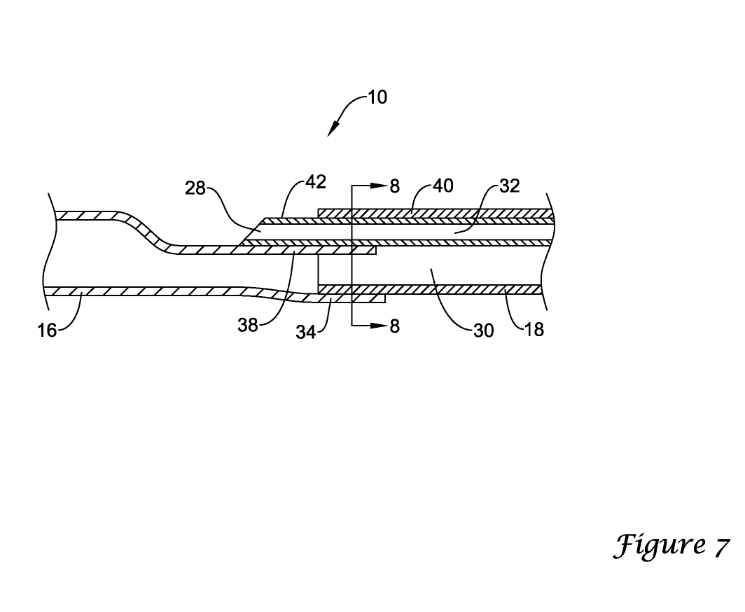

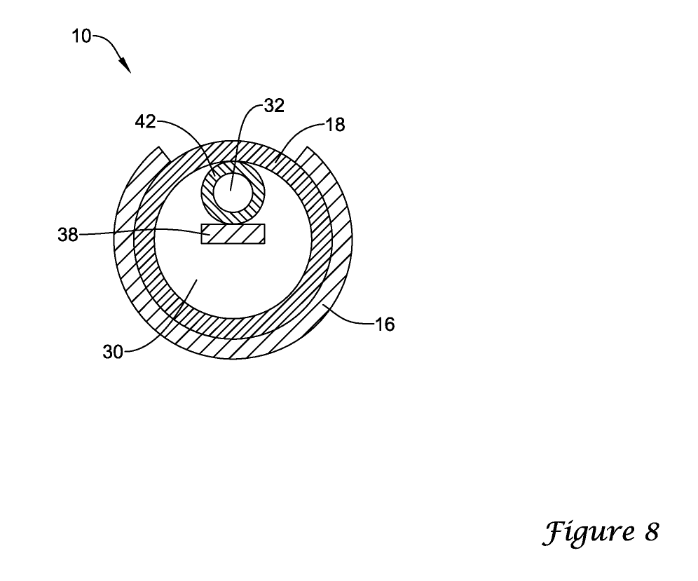

A proximal end 40 of distal shaft portion 18 may be disposed within the enlarged distal end 34 of midshaft portion 16 as shown in FIG. 7. In doing so, tongue 38 may be pressed inward and form a shelf or ledge. A distal inner tube 42 may be disposed within distal shaft portion 18 and may rest upon the ledge formed by tongue 38. Distal inner tube 42 may ultimately form guidewire lumen 32 as described in more detail below. The arrangement of distal inner tube 42 relative to tongue 38, midshaft portion 16, and distal shaft portion 18 can also be seen in FIG. 8.

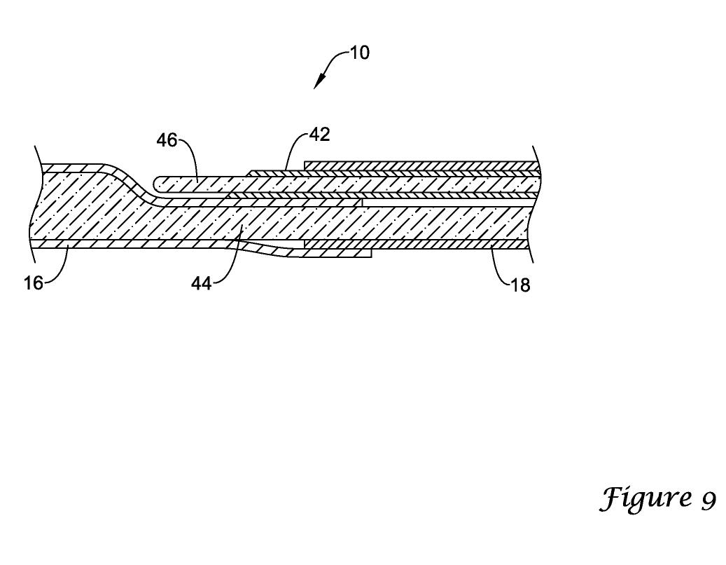

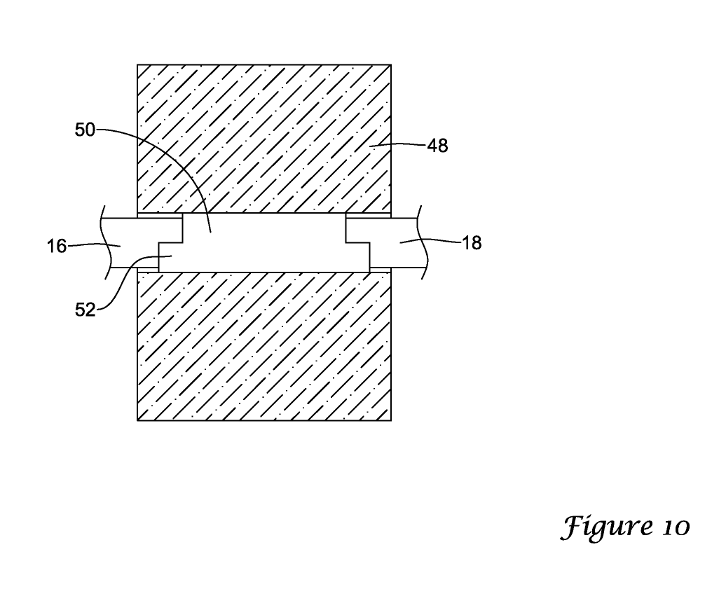

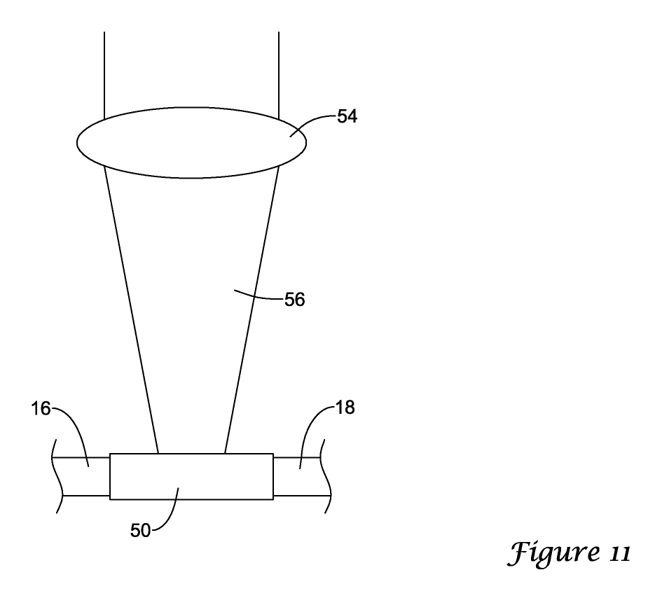

When suitably arranged, a first mandrel 44 may be inserted within a portion of distal shaft portion 18 and midshaft portion 16 as shown in FIG. 9. Likewise, a second mandrel 46 may be inserted within distal inner tube 42. With mandrels 44/46 in place, midshaft portion 16 and distal shaft portion 18 may be disposed within a compression fixture 48 as shown in FIG. 10. A sleeve 50 may be disposed over a region of midshaft portion 16 and distal shaft portion 18. Sleeve 50 may include one or more flanking ears 52, which may aid in removal of sleeve 50 upon completion of the manufacturing process. Finally, heat may be applied to sleeve 50. This may include the use of a lens 54 to focus heat (e.g., laser energy 56) onto sleeve 50 as depicted in FIG. 11. When heated, midshaft portion 16, distal shaft portion 18, and distal inner tube 42 may melt together. Mandrels 44/46 can be removed, thereby defining inflation lumen 30 and guidewire lumen 32, respectively, and the result may be the formation of catheter shaft 12 as shown in FIGS. 1-3.

The materials that can be used for the various components of catheter 10 may include those commonly associated with medical devices. For simplicity purposes, the following discussion makes reference to catheter shaft 12 and other components of catheter 10. However, this is not intended to limit the devices and methods described herein, as the discussion may be applied to other similar tubular members and/or components of tubular members or devices disclosed herein.

Catheter shaft 12 and/or other components of catheter 10 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of catheter shaft 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of catheter 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of catheter 10 to achieve the same result.

In some embodiments, a degree of Magnetic Resonance Imaging (MRI) compatibility is imparted into catheter 10. For example, catheter shaft 12, or portions thereof, may be made of a material that does not substantially distort the image and create substantial artifacts (i.e., gaps in the image). Certain ferromagnetic materials, for example, may not be suitable because they may create artifacts in an MRI image. Catheter shaft 12, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of catheter shaft 12 that may define a generally smooth outer surface for catheter 10. In other embodiments, however, such a sheath or covering may be absent from a portion of all of catheter 10, such that catheter shaft 12 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the catheter 10 (including, for example, the exterior surface of catheter shaft 12) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of catheter shaft 12, or other portions of catheter 10. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

The entire disclosures of U.S. Pat. Nos. 6,409,863, 5,156,594, 5,720,724, 6,361,529, and 6,475,187 are herein incorporated by reference.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A balloon catheter, comprising:
a proximal shaft;
a midshaft attached to the proximal shaft, wherein the midshaft has an outer wall;
a distal shaft attached to the midshaft;
a balloon coupled to the distal shaft;
wherein an inflation lumen is defined that extends from the proximal shaft, through the midshaft, and into the distal shaft, the inflation lumen being in fluid communication with the balloon; and
a support member attached to the outer wall of the midshaft.

2. The balloon catheter of claim 1, wherein the midshaft has a guidewire port formed therein.

3. The balloon catheter of claim 2, wherein the support member extends proximally of the guidewire port.

4. The balloon catheter of claim 3, wherein the support member extends distally of the guidewire port.

5. The balloon catheter of claim 1, wherein the support member is a metallic rod.

6. The balloon catheter of claim 1, further comprising one or more additional support members.

7. The balloon catheter of claim 1, wherein the midshaft includes a supporting braid, a supporting coil, or both incorporated into a wall surface of the midshaft.

8. A balloon catheter, comprising:
a proximal shaft;
a midshaft attached to the proximal shaft, wherein the midshaft has an outer wall;
a distal shaft attached to the midshaft;
a balloon coupled to the distal shaft;
wherein a guidewire port is defined in the midshaft that provides access to a guidewire lumen; and
a support member attached to the outer wall of the midshaft, the support member being a longitudinally extending rod with a proximal end disposed proximally of the guidewire port and a distal end disposed distally of the guidewire port.

9. The balloon catheter of claim 8, further comprising one or more additional support members.

10. The balloon catheter of claim 9, wherein two support members are attached to the outer wall of the midshaft.

11. The balloon catheter of claim 10, wherein the two support members are disposed on opposite sides of the midshaft.

12. The balloon catheter of claim 8, wherein the support member is attached to the outer wall of the midshaft with an adhesive.

13. The balloon catheter of claim 8, wherein the support member is partially embedded within the outer wall of the midshaft.

14. The balloon catheter of claim 8, wherein the support member includes stainless steel.

15. The balloon catheter of claim 8, wherein the support member includes polyimide or polyetheretherketone.

16. A method for manufacturing a balloon catheter, the method comprising:
providing a catheter shaft including a proximal shaft, a midshaft attached to the proximal shaft, and a distal shaft attached to the midshaft;
wherein a guidewire port is defined in the midshaft, the guidewire port providing access to a guidewire lumen formed in the catheter shaft;
providing a support member; and
attaching the support member to the midshaft, the support member extending both proximally and distally of the guidewire port.

17. The method of claim 16, wherein attaching the support member to the midshaft includes attaching the support member to the midshaft with an adhesive.

18. The method of claim 16, wherein attaching the support member to the midshaft includes heating the midshaft and partially embedding the support member within the midshaft.

19. The method of claim 16, wherein attaching the support member to the midshaft includes disposing the support member adjacent to the midshaft, disposing a heat shrink tube about the support member and the midshaft, and heating the heat shrink tube.

20. The method of claim 19, wherein heating the heat shrink tube includes heating with a laser.
